(19) United States
(12) Patent Application Publication
(10) Pub. No.: US 2024/0279170 A1
(43) Pub. Date: Aug. 22, 2024

(54) METHOD FOR PREPARING THIURAM DISULFIDES

(71) Applicant: MLPC INTERNATIONAL, Rion-Des-Landes (FR)

(72) Inventors: Kévin FOURMY, Rion-Des-Landes (FR); Anaïs VIGNAULT, Rion-Des-Landes (FR)

(73) Assignee: MLPC INTERNATIONAL, Rion-Des-Landes (FR)

(21) Appl. No.: 18/565,390

(22) PCT Filed: May 31, 2022
(86) PCT No.: PCT/EP2022/064820

§ 371 Date: Nov. 29, 2023

(30) Foreign Application Priority Data
Jun. 1, 2021 (FR) 2105743

(51) Int. Cl. C07C 333/32; C08K 5/40

(52) U.S. Cl. CPC C07C 333/32; C08K 5/40

(57) ABSTRACT

The present invention relates to a process for preparing a thiuram disulfide, comprising a step of simultaneously adding an oxidizing composition and a dithiocarbamate salt composition to a reactor, said reactor already comprising a solvent S₁. The present invention also relates to a thiuram disulfide obtainable by the process as according to the invention, and to the use thereof as a vulcanization accelerator.

The present invention relates to a process for preparing thiuram disulfides, to the thiuram disulfides obtainable by said process, and to the uses thereof, in particular as vulcanization accelerators and/or as sulfur donors. Tetrabenzylthiuram disulfide (referred to hereinafter as TBzTD) is particularly preferred in the context of the present invention.

Thiuram disulfides are known as vulcanization accelerating agents, particularly for the vulcanization of various types of rubber such as tires or industrial rubbers. They are also known to be sulfur donors which confer good heat resistance due to the mono- or disulfide sulfur bridges that they create. They are particularly useful in the vulcanization of natural rubber, butadiene rubbers, butyl rubbers, EPDM (for ethylene-propylene-diene monomer), latex, butadiene-acrylonitrile copolymer (or nitrile rubber or NBR for Nitrile Butadiene Rubber) or styrene-butadiene copolymer (or SBR for Styrene Butadiene Rubber). Their use makes it possible to improve the efficiency and/or the speed of the vulcanization process, without affecting the properties of the final rubber.

However, one of the problems associated with their use in vulcanization is the generation of volatile nitrosamines when they are prepared from light secondary amines such as diethylamine or dibutylamine. Specifically, volatile nitrosamines are degradation products of these accelerators that it is desired to avoid since they are carcinogenic. Thus, some thiuram disulfides are prepared from heavier amines and therefore generate nitrosamines that are not volatile (they are called “nitrosamine-safe”). In particular, they enable effective acceleration of the vulcanization process while being more environmentally friendly and without risk compared to thiuram disulfides derived from light amines. Among the “nitrosamine-safe” thiuram disulfides, mention may be made of tetrabenzylthiuram disulfide (TBzTD), tetrakis(2-ethylhexyl)thiuram disulfide (TOTD) or tetraisobutylthiuram disulfide (TiBTD).

Thiuram disulfides, whether “nitrosamine-safe” or not, must meet precise specifications with a view to their use as vulcanization accelerating agents. Good purity is thus sought in order to guarantee good activity of these products. In the case of thiuram disulfides that are solid at ambient temperature, such as TBzTD, a controlled melting point is also sought.

There is therefore a need for a process for preparing thiuram disulfides which makes it possible to obtain a product with a yield and quality that are satisfactory, or even improved compared to the known processes. There is also a need for a process for preparing thiuram disulfides which is simple to implement industrially.

There is also a need for thiuram disulfides which are as pure as possible, and/or which have controlled physicochemical properties.

One objective of the present invention is to provide a process for preparing thiuram disulfides which is easy to implement and/or has a good yield, preferably an improved yield compared to the known processes.

One objective of the present invention is also to provide thiuram disulfides having a satisfactory purity, or even a purity greater than the thiuram disulfides produced to date, preferably at least 99%.

Another objective of the present invention is to provide thiuram disulfides with a controlled melting point, preferably a TBzTD having a final melting point of greater than or equal to 135° C., preferably strictly greater than 135° C.

The present invention meets all or part of the above objectives.

Thus, the present invention relates to a process for preparing a thiuram disulfide, comprising a step of simultaneously adding an oxidizing composition and a composition comprising a dithiocarbamate salt to a reactor, said reactor already comprising a solvent S₁.

The synthesis of thiuram disulfides conventionally comprises the following steps:
preparation of a dithiocarbamate salt, then
oxidation of said salt to thiuram disulfide.

Said synthesis can be illustrated by that of TBzTD according to the scheme below:

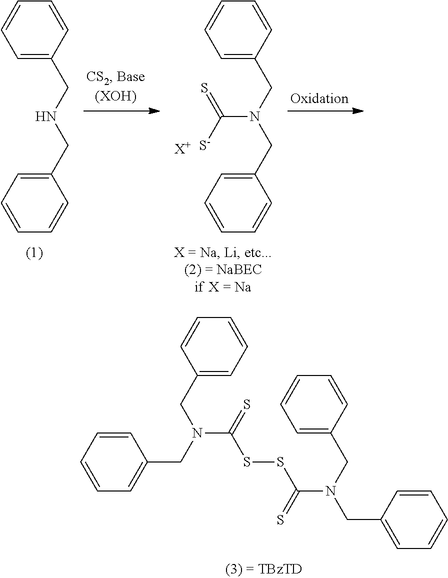

In the case of the TBzTD schematized above, the sodium salt of dibenzyldithiocarbamate (2) (referred to as NaBEC) is prepared from dibenzylamine (1) and carbon disulfide, in the presence of a base, preferably in the presence of sodium hydroxide. The NaBEC obtained is then oxidized to form TBzTD (3), which precipitates at the end of the reaction.

This conventional synthesis route is described for example in documents U.S. Pat. No. 6,465,691B1 and U.S. Pat. No. 4,468,526. Document CN 1827596A describes a preparation process characterized by an oxidation step with simultaneous addition of hydrogen peroxide and sulfuric acid to a reactor comprising NaBEC. The TBzTD obtained is in the form of a slightly yellow solid have a degree of purity of 97%. No melting point of the TBzTD obtained is given.

The present inventors have surprisingly discovered that a particular implementation of the step of oxidizing the dithiocarbamate salt makes it possible to obtain a thiuram disulfide with a high purity, in particular with a purity of greater than or equal to 99% (% by mass); the purity being able to be determined in a conventional manner by HPLC.

Such a purity notably enables controlled physicochemical properties of the thiuram disulfide, such as the melting point, preferably the final melting point.

In particular, the TBzTD obtained with the process according to the invention has a melting point, preferably a final melting point, of greater than or equal to 135° C., and preferentially strictly greater than 135° C. “Final melting point” in particular means the temperature at which the melting of the thiuram disulfide is complete (when there is no longer any solid form). The, in particular final, melting point may be measured in a conventional manner by DSC (Differential Scan Calorimetry). The melting point, more specifically the final melting point, is in particular determined at atmospheric pressure (or around 1013.25 hPa).

In particular, the TBzTD obtained according to the invention is white in color, without yellowing. It may be easy to recover and to use: it is easy to filter and does not compact.

The process as according to the invention also makes it possible to obtain a yield of greater than or equal to 90%, preferably greater than or equal to 95%, or even greater than or equal to 97%.

The process according to the invention may be implemented in a simple manner, at mild temperatures, and preferably with water as solvent. It is therefore economically viable and environmentally friendly. It may be performed batchwise or continuously, preferably batchwise.

### Definitions

The term “—(C₁-C₁₅)alkyl” in particular denotes monovalent saturated aliphatic hydrocarbons which may be linear or branched and which comprise from 1 to 15 carbon atoms. Preferably, the alkyls comprise from 1 to 8 carbon atoms. Mention may for example be made of methyl, ethyl, n-propyl, isopropyl, n-butyl, isobutyl, sec-butyl or tert-butyl. “Branched” means that an alkyl group is substituted on the main alkyl chain.

The term “—(C₆-C₁₀)aryl” in particular denotes monocyclic, bicyclic or tricyclic monovalent aromatic hydrocarbon compounds comprising from 6 to 10 carbon atoms, in particular phenyl and naphthyl.

The term “—(C₃-C₁₀)cycloalkyl” in particular denotes monocyclic or bicyclic monovalent saturated aliphatic hydrocarbons comprising from 3 to 10 carbon atoms, such as cyclopropyl, cyclobutyl, cyclopentyl or cyclohexyl.

“—(C₄-C₁₀)heteroaryl” in particular means an aryl as defined above, comprising between 4 and 10 carbon atoms and comprising at least one heteroatom; for example a furanyl or a pyridinyl.

“—(C₁-C₁₅)alkylene” in particular means an alkyl radical as defined above but divalent.

“Heteroatom” in particular means an atom chosen from O, N, S, Si, P and halogens, preferably O, N or S.

“Unsaturation” in particular means a double or triple bond between two carbon atoms.

The present invention therefore relates to a process for preparing a thiuram disulfide, comprising a step of simultaneously adding an oxidizing composition and a composition comprising a dithiocarbamate salt to a reactor, said reactor already comprising a solvent S₁. Said addition step enables the oxidation of the dithiocarbamate salt to thiuram disulfide.

More particularly, the present invention relates to a process for preparing a thiuram disulfide of general formula (I) below:

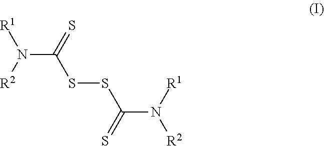

in which the radicals R₁ and R₂ are chosen independently of one another from the group consisting of:
   —(C₁-C₅)alkyls,
   —(C₃-C₁₀)cycloalkyls, —(C₁-C₁₅)alkylene-(C₃-C₁₀)cycloalkyls,
   —(C₆-C₁₀)aryls, —(C₁-C₅)alkylene-(C₆-C₁₀)aryls, and
   —(C₄-C₁₀)heteroaryls,
it being possible for said alkyl or alkylene chain to be interrupted by one or more oxygen atoms —O— and/or by one or more —N(R₅)— groups where R₅ is a —(C₁-C₅)alkyl; and it being possible for said alkyl or alkylene chain to comprise one or more unsaturations; said process comprising a step of simultaneously adding an oxidizing composition and a composition comprising a dithiocarbamate salt of general formula (II) below to a reactor:

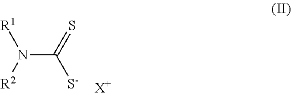

in which
R₁ and R₂ are as defined for the general formula (I) and X is chosen from the group consisting of alkali metals, alkaline-earth metals and ammoniums;
said reactor already comprising a solvent S₁.

Preferably, the oxidizing composition and the composition comprising a dithiocarbamate salt are simultaneously introduced into the reactor above the surface of the solvent S₁ present. More particularly, said compositions are introduced at the top of the reactor (that is to say through the top of the reactor), whereas the solvent S₁ is placed at the bottom of the reactor.

The oxidizing composition and the composition comprising a dithiocarbamate salt may each be introduced separately or be mixed beforehand before introduction into the reactor. They are preferably introduced separately. Said introduction may take place by injection.

“Reaction medium” in particular means the medium contained in said reactor and preferably comprising said oxidizing composition, said dithiocarbamate salt composition and said solvent S₁.

The addition step may be carried out at a temperature of between 5° C. and 60° C., preferably between 15° C. and 25° C., for example between 18° C. and 20° C.

During said addition step, the reaction medium may be stirred, for example using blades in the reactor. If necessary, the stirring may continue after the addition step.

During the addition step, the pH of the reaction medium may be neutral or basic. It may therefore be between 7 and 14. It is preferably greater than 7 and even more preferentially between 8 and 12, for example between 8 and 10. The pH may be monitored in a conventional manner by a pH meter.

The pH of the reaction medium may advantageously be kept constant throughout the addition step, particularly by controlling the [dithiocarbamate salt/oxidizer] molar ratios introduced. Specifically, the dithiocarbamate salt composition and the oxidizing composition may be introduced into the reactor at different molar flow rates, particularly with a dithiocarbamate salt molar flow rate greater than that of the oxidizer. In particular, the [dithiocarbamate salt/oxidizer] molar ratio introduced into the reactor is between 1 and 4, preferably between 1.5 and 3, more preferentially between 1.5 and 2.5. Preferably, the [dithiocarbamate salt/oxidizer] molar ratio introduced into the reactor is greater than or equal to 2.

In particular, the addition of the dithiocarbamate salt composition may end at the same time or before the end of the addition of the oxidizing composition. Advantageously, even though the addition of the oxidizing and dithiocarbamate salt compositions is simultaneous, the addition of the dithiocarbamate salt composition may end before the end of the addition of the oxidizing composition. In other words, the pouring of the dithiocarbamate salt composition may end before the pouring of the oxidizing composition. In this case, it is understood that the pouring of the two compositions is simultaneous until the end of the pouring of the dithiocarbamate salt composition. This may for example be obtained by virtue of a [dithiocarbamate salt/oxidizer] molar ratio introduced into the reactor of strictly greater than 2, for example between strictly greater than 2 and 4.

The addition step lasts for example between 3 h and 10 h, preferably between 4 h and 6 h.

According to one embodiment, the addition step is carried out without a catalyst.

At the end of the addition step, in the reaction medium, the [dithiocarbamate salt/oxidizer] molar ratio may be between 1.5 and 3, preferably between 1.8 and 2.5. At the end of the addition step, the pH may be acidic, for example between 2 and 5, preferably between 3 and 4.

According to one embodiment, said thiuram disulfide (in particular the TBzTD) thus obtained precipitates in said solvent S₁. It is then possible to recover it by filtration, then optionally wash it with water and dry it.

### Thiuram Disulfide:

Preferably, the thiuram disulfides are of general formula (I) as defined above.

Preferably, R₁ and R₂ are chosen from —(C₁-C₅)alkyls and —(C₁-C₅)alkylene-(C₆-C₁₀)aryls. In particular, R₁ and R₂ are chosen from —(C₁-C₁₀)alkyls and —(C₁-C₁₀)alkylene-(C₆-C₁₀)aryls.

According to one embodiment, R₁ and R₂ may be different or identical; preferably, they are identical.
The thiuram disulfide may be chosen from the group consisting of:
tetrabenzylthiuram disulfide (TBzTD, CAS No.: 10591-85-2),
tetramethylthiuram disulfide (TMTD, CAS No.: 97-74-5),
tetraethylthiuram disulfide (TETD, CAS No.: 97-77-8),
tetrabutylthiuram disulfide (TBTD, CAS No.: 1634-02-2),
tetrakis(2-ethylhexyl)thiuram disulfide (TOTD, CAS No.: 37437-21-1), and
tetraisobutylthiuram disulfide (TiBTD, CAS No.: 3064-73-1).

In particular, the thiuram disulfide is chosen from the group consisting of:
tetrabenzylthiuram disulfide (TBzTD),
tetrakis(2-ethylhexyl)thiuram disulfide (TOTD), and
tetraisobutylthiuram disulfide (TiBTD).

Preferably, the thiuram disulfide is the tetrabenzylthiuram disulfide (TBzTD) of the formula below:

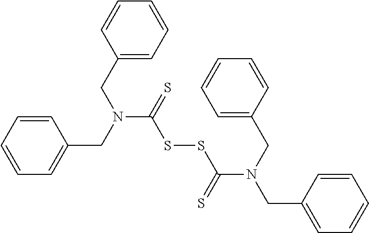 [SMILES: S=C(SSC(=S)N(Cc1ccccc1)Cc1ccccc1)N(Cc1ccccc1)Cc1ccccc1]

### Dithiocarbamate Salts:

The dithiocarbamate salts are preferentially of general formula (II) as defined above, where R₁ and R₂ are as defined for the general formula (I) and X is chosen from the group consisting of alkali metals, alkaline-earth metals and ammoniums.

“Alkali metals” in particular means lithium, sodium, potassium, rubidium and cesium. Particular preference is given to sodium and potassium and most preferred is sodium.

“Alkaline-earth metals” in particular means beryllium, magnesium, calcium, strontium, barium and radium, preferably magnesium and calcium.

“Ammoniums” in particular means the NH₄⁺ ions originating from gaseous ammonia (NH₃) or aqueous ammonia (NH₄OH).

Most preferably, X is sodium.

Preferably, the dithiocarbamate salt used is chosen from the group consisting of:
sodium salt of dibenzylamine dithiocarbamate (also referred to as NaBec),
sodium salt of dimethylamine dithiocarbamate,
sodium salt of diethylamine dithiocarbamate,
sodium salt of dibutylamine dithiocarbamate,
sodium salt of diisobutylamine dithiocarbamate,
sodium salt of bis(2-ethylhexyl)amine dithiocarbamate.

Most particularly preferably, the dithiocarbamate salt is NaBec.

### Composition Comprising a Dithiocarbamate Salt:

Said dithiocarbamate salt is in particular in solution in order to carry out said process according to the invention. Thus, the dithiocarbamate salt composition may be a solution, preferably an aqueous, alcoholic or aqueous/alcoholic solution. The solvent may be chosen from water; alcohols such as methanol, ethanol, isopropanol, n-propanol, n-butanol, tert-butanol and amyl alcohol; or mixtures thereof. Preferably, the solvent is water.

Preferentially, the dithiocarbamate salt composition comprises from 15% to 95%, preferably from 50% to 90%, preferentially from 70% to 90%, by weight of solvent relative to the total weight of said composition.

Preferentially, the dithiocarbamate salt composition comprises from 5% to 85% by weight, preferably from 10% to 50%, preferentially from 10% to 30%, by weight of dithiocarbamate salt relative to the total weight of said composition.

Preferably, said composition is an aqueous solution of dithiocarbamate salt, preferably of NaBEC, comprising, or even consisting of:
i) from 50% to 90%, preferably from 70% to 90%, by weight of water relative to the total weight of said solution; and
ii) from 10% to 50%, preferably from 10% to 30%, by weight of dithiocarbamate salt, preferably of NaBEC, relative to the total weight of said solution.

### Oxidizing Composition:

“Oxidizing composition” means any composition comprising at least one oxidizer (or oxidizing agent) which makes it possible to oxidize the dithiocarbamate salt so as to obtain a thiuram disulfide. Such an oxidizer is preferentially hydrogen peroxide or a peroxide derivative originating from hydrogen peroxide. It may be a derivative resulting from the mixing of hydrogen peroxide with an acid. Said acid may be of any type, mineral or organic. Mention may for example be made of sulfuric acid (H₂SO₄), hydrochloric acid (HCl), carboxylic acids such as acetic acid or any other acid which is soluble in a hydrogen peroxide solution. Particular preference is given to sulfuric acid.

Thus, for example, mixing hydrogen peroxide with sulfuric acid forms peroxymonosulfuric acid.

Said composition may be a solution, preferably an aqueous solution and more preferentially an aqueous solution of hydrogen peroxide.

Said composition may be a composition comprising hydrogen peroxide, water and optionally an acid (in particular as defined above). It is possible to pour the acid separately into said reactor. However, when the acid is present, preference is given to preparing an oxidizing composition comprising said acid before addition to the reactor.

Preferably, the oxidizing composition is an aqueous solution of hydrogen peroxide and sulfuric acid (or an aqueous solution of peroxymonosulfuric acid).

In particular, the oxidizer is not oxygen (O₂).

Preferentially, said oxidizing composition comprises, or even consists of, per one molar equivalent of said dithiocarbamate salt:
between 0.40 and 0.65, more preferentially between 0.5 and 0.55, molar equivalent of hydrogen peroxide; and
optionally between 0.40 and 0.65, more preferentially between 0.5 and 0.55, molar equivalent of acid.

In particular, the amount of hydrogen peroxide is between 0.5% and 20% by weight, preferably between 1% and 10% by weight, more preferentially between 1% and 5% by weight, relative to the total weight of said oxidizing composition.

In particular, the amount of acid is between 2% and 40% by weight, preferably between 5% and 20% by weight, more preferentially between 5% and 10% by weight, relative to the total weight of said oxidizing composition.

In particular, the amount of solvent, preferably water, is between 0 and 95% by weight, preferably between 60% and 80% by weight, relative to the total weight of said oxidizing composition.

The oxidizing composition may be prepared in a conventional manner, for example by mixing the ingredients in a tank, or continuously by means of a static mixer.

### Solvent S₁ Present in the Reactor:

The fact that “the reactor already comprises a solvent S₁” in particular means that when the oxidizing composition and the dithiocarbamate salt composition are simultaneously added to the reactor, the latter already comprises the solvent S₁ (this is also referred to as “a solvent bottom”). The solvent S₁ may be the same or different from the solvent(s) used for the oxidizing composition and/or the dithiocarbamate salt composition. In particular, the solvent S₁, that of the oxidizing composition and that of the dithiocarbamate salt composition is water.

Said solvent S₁ may be an aqueous, alcoholic, aqueous/alcoholic or organic solvent. Thus, the solvent S₁ may be chosen from water; alcohols such as methanol, ethanol, isopropanol, n-propanol, n-butanol, tert-butanol and amyl alcohol; organic solvents such as toluene and dichloromethane; and mixtures thereof, in particular mixtures of water and alcohol(s). Preferably, the solvent S₁ is water.

In particular, the amount by weight of solvent S₁ already present in the reactor is between 5% and 50% by weight, preferably between 10% and 40% by weight, more preferentially between 20% and 40% by weight, for example between 20% and 30% by weight, relative to the total weight of the oxidizing and dithiocarbamate salt compositions which are poured into the reactor.

Said solvent S₁ may also comprise a surfactant, preferably a nonionic surfactant. Examples of nonionic surfactants that may be mentioned are alkoxylated fatty acids, in particular ethoxylated fatty acids, ethoxylated alcohols, ethoxylated sorbitan esters, and castor oil ethoxylates. In particular, mention may be made of the following surfactants: Tergitol®15-S-5, Tergitol®15-S-7, Tergitol®15-S-9, Tergitol®15-S-12, Ecosurf®SA-4, Ecosurf®SA-7, Ecosurf®SA-9, Rhodasurf® 870H20, Synative® RPE 2520, Breox® 50 A 20, Breox® 50 A 50, Breox® 50 A 140 or Breox® 43 A 1000.

The surfactant may in particular be added to the solvent S₁ before the step of simultaneously adding the oxidizing and dithiocarbamate salt compositions.

Preferably, the amount of surfactant in said solvent S₁ is between 0.005% and 1%, preferably between 0.01% and 0.5%, more preferentially between 0.05% and 0.5%, by weight relative to the theoretical total weight of thiuram to be obtained at the end of the addition step.

The process for synthesizing the thiuram disulfide may comprise the following steps:
i) preparing a composition comprising a dithiocarbamate salt, then
ii) simultaneously adding an oxidizing composition and the composition comprising the dithiocarbamate salt obtained in step i) to a reactor, said reactor already comprising a solvent S₁,
iii) optionally recovering the thiuram disulfide, preferably by filtration.

Step i) may be carried out by reacting an amine, preferably a secondary amine corresponding to the desired thiuram disulfide, and CS₂, preferably in the presence of a base. The base is in particular a base of type X—OH, where X is chosen from alkali metals, alkaline-earth metals or NH₄; in particular, X is sodium or lithium.

The dithiocarbamate salts are thus obtained from a conventional and widely described synthesis using for example one molar equivalent of the amine, one molar equivalent of CS₂ and one molar equivalent of sodium hydroxide (50% by mass). In particular, the dithiocarbamate salt is obtained by adding CS₂ to the mixture of the amine and the base, preferably at a temperature of between 20° C. and 40° C. A composition comprising a dithiocarbamate salt is thus obtained.

Step ii) corresponds to the simultaneous addition step as according to the invention and defined above.

Preferably, steps i) and ii) do not take place in the same reactor, but in two different reactors. Use may be made of reactors of batch type or tubular reactors of continuous mixer type for step i) and/or ii). Step ii) is preferentially carried out in a batch reactor.

Step iii) of recovering the thiuram disulfide may be effected by any known means, and in particular by filtration when said thiuram disulfide precipitates in the solvent S₁. It is then optionally possible to wash it with water and dry it.

The present invention also relates to a thiuram disulfide, preferably of general formula (I) as defined above, obtainable by the preparation process as according to the invention. The present invention also relates to a thiuram disulfide, preferably of general formula (I) as defined above, obtained or directly obtained by the preparation process as according to the invention. Preferably, said thiuram disulfide is TBzTD.

The present invention relates to a TBzTD having a melting point, in particular a final melting point, of between 134° C. and 137° C., preferably between 135.5° C. and 137° C., and/or a purity of greater than or equal to 99% (% by mass). Preferably, said TBzTD has a melting point, in particular a final melting point, of strictly greater than 135° C., more preferentially strictly greater than 135.5° C., and/or a purity of greater than or equal to 99% (% by mass). In particular, the TBzTD has a final melting point of greater than or equal to 136° C., for example of 137° C. Said TBzTD is notably obtainable (or obtained or directly obtained) by the process as according to the invention.

The present invention also relates to a composition comprising at least 99% by weight of TBzTD, said TBzTD having a melting point, in particular a final melting point, of strictly greater than 135° C., more preferentially strictly greater than 135.5° C. In particular, the TBzTD has a final melting point of greater than or equal to 136° C. Said TBzTD is notably obtainable (or obtained or directly obtained) by the process as according to the invention.

The present invention also relates to the use of thiuram disulfide as according to the invention as a vulcanization accelerator or an additive for lubricants, preferably an accelerator for the vulcanization of natural rubber, butadiene rubbers, butyl rubbers, EPDM (ethylene-propylene-diene monomer), latex, butadiene-acrylonitrile copolymer (or nitrile rubber or NBR for Nitrile Butadiene Rubber) or styrene-butadiene copolymer (or SBR for Styrene Butadiene Rubber).

### DESCRIPTION OF THE FIGURES

FIG. 1 corresponds to one embodiment of the process as according to the invention. A composition comprising a dithiocarbamate salt of formula (II) and an oxidizing composition are introduced into a reactor with blades and comprising a solvent bottom S₁, simultaneously and above the surface of the solvent S₁. A thiuram disulfide of formula (I) is obtained and recovered at the bottom of the reactor.

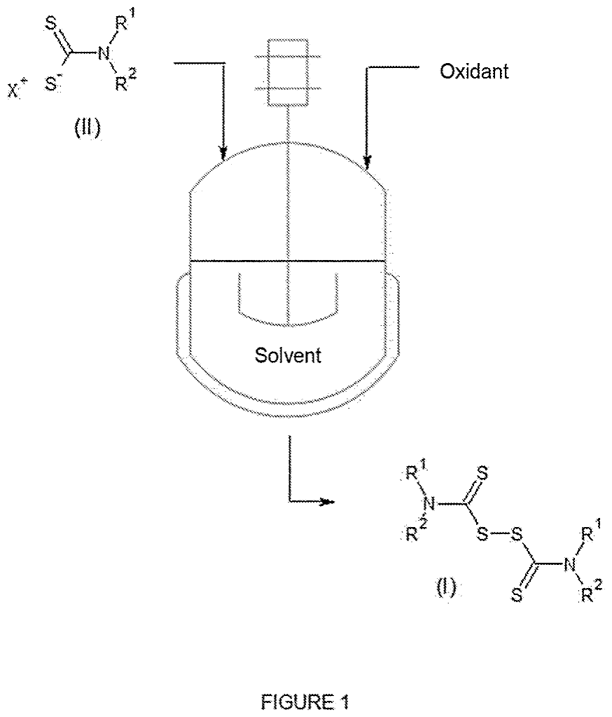

The expression “between X and X” includes the stated endpoints, unless mentioned otherwise.

The examples below are given for illustrative purposes and do not limit the present invention.

### EXAMPLES

### Example 1: Synthesis of TBzTD According to the Invention

### I. Preparation of the Oxidizing Composition and of the NaBEC Composition

139.2 g of water is introduced into a 500 ml flask. 35.1 g (0.15 mol, 1 eq.) of 43% by mass sulfuric acid is added. 15.3 g (0.16 mol, 1.05 eq.) of 35% by mass hydrogen peroxide is then added. Homogenization is performed at ambient temperature. 367.2 g (0.3 mol, 2 eq.) of around 24.8% by mass NaBEC is introduced into another 500 ml flask.

### II. Addition Step According to the Invention

The reactor already contains 197 g of water stirred at 18° C. and containing 0.1% by mass of the surfactant RHODASURFO 870 H 20.

Using a first pump, the NaBEC composition is injected into the reactor.

Using a second pump, the oxidizing composition is simultaneously injected into the reactor.

The flow rate at which the NaBEC composition is introduced into the reactor is 1.31 g/min, or a molar flow rate of pure NaBEC of 1.1 mmol/min.

The flow rate at which the oxidizing composition is introduced into the reactor is 0.63 g/min, or a molar flow rate of pure oxidizer of 0.5 mmol/min. This flow rate being calculated by theoretically assuming the formation of 0.15 mol of peroxymonosulfuric acid oxidizer in the mixture prepared here from 0.15 mol of sulfuric acid and 0.16 mol of hydrogen peroxide.

The ratio of the [NaBEC/ox] molar flow rates is therefore 2.2, and the pH is between 8 and 10 until the end of the injection of the NaBEC.

At the end of the addition step, the reactor is held at 18° C. for 5 min with stirring, then is drained.

The suspension obtained is filtered on a frit, then washed with water.

The TBzTD is obtained in the form of a wet white solid, which has precipitated. It is recovered and placed in an oven overnight at 40° C.

The results obtained are as follows:
Yield (moles of TBzTD obtained/moles of theoretical TBzTD): 99.5%
TBzTD purity (% by mass) (by HPLC): 99.4%
TBzTD melting T°: 136° C. (final melting point: 137° C.)

### Example 2: Comparative Synthesis Example of TBzTD without Simultaneous Addition

### I. Preparation of the Oxidizing Composition

179.3 g of water is introduced into a 500 ml flask. 28.3 g (0.12 mol, 1 eq.) of 43% by mass sulfuric acid is added. 11.7 g (0.12 mol, 1 eq.) of 35% by mass hydrogen peroxide is then added. Homogenization is performed at ambient temperature.

### II. Addition Step

288.4 g (0.24 mol, 2 eq.) of around 24.8% by mass NaBEC is introduced into a reactor. 76.56 g of water and 0.1% (0.07 g) by mass of the surfactant RHODASURFO 870 H 20 are then added at 18° C. and with stirring.

Using a peristaltic pump, the oxidizing composition is introduced into the reactor, already containing the NaBEC, at a flow rate of 0.61 g/min.

At the end of the step of adding the oxidizing composition, the reactor is held at 18° C. for 5 min with stirring, then is drained.

The suspension obtained is filtered on a frit, then washed with water.

The TBzTD is obtained in the form of a wet white solid, which has precipitated. It is recovered and placed in an oven overnight at 40° C.

The results obtained are as follows:
Yield (moles of TBzTD obtained/moles of theoretical TBzTD): 80.1%
TBzTD purity (% by mass) (by HPLC): 92.7%
TBzTD melting T°: 128.5° C. (final melting point: 131.1° C.).

1. A process for preparing a thiuram disulfide, comprising a step of simultaneously adding an oxidizing composition and a composition comprising a dithiocarbamate salt to a reactor, said reactor already comprising a solvent S₁, preferably water.

2. The process for preparing a thiuram disulfide according to claim 1, in which said thiuram disulfide is of general formula (I) below: 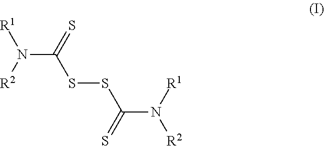
in which the radicals R₁ and R₂ are chosen independently of one another from the group consisting of:
—(C₁-C₁₅)alkyls,
—(C₃-C₁₀)cycloalkyls, —(C₁-C₁₅)alkylene-(C₃-C₁₀)cycloalkyls,
—(C₆-C₁₀)aryls, —(C₁-C₁₅)alkylene-(C₆-C₁₀)aryls, and
—(C₄-C₁₀)heteroaryls,
it being possible for said alkyl or alkylene chain to be interrupted by one or more oxygen atoms —O— and/or by one or more —N(R₅)— groups where R₅ is a —(C₁-C₁₅)alkyl; and
it being possible for said alkyl or alkylene chain to comprise one or more unsaturations.

3. The process for preparing a thiuram disulfide according to claim 1, in which the thiuram disulfide is chosen from the group consisting of:
tetrabenzylthiuram disulfide (TBzTD),
tetramethylthiuram disulfide (TMTD),
tetraethylthiuram disulfide (TETD),
tetrabutylthiuram disulfide (TBTD),
tetrakis(2-ethylhexyl)thiuram disulfide (TOTD), and
tetraisobutylthiuram disulfide (TiBTD).

4. The process for preparing a thiuram disulfide according to claim 1, in which the thiuram disulfide is tetrabenzylthiuram disulfide.

5. The process for preparing a thiuram disulfide according to claim 1, in which the oxidizing composition comprises hydrogen peroxide, water and optionally an acid.

6. The process for preparing a thiuram disulfide according to claim 1, in which the solvent S₁ is chosen from water; alcohols such as methanol, ethanol, isopropanol, n-propanol, n-butanol, tert-butanol and amyl alcohol; organic solvents such as toluene and dichloromethane; and mixtures thereof.

7. The process for preparing a thiuram disulfide according to claim 1, in which during the addition step, the [dithiocarbamate salt/oxidizer] molar ratio introduced into the reactor is between 1 and 4, preferably between 1.5 and 3, more preferentially between 1.5 and 2.5.

8. The process for preparing a thiuram disulfide according to claim 1, in which during the addition step, the pH is between 7 and 14, preferably between 8 and 12, more preferably between 8 and 10.

9. The process for preparing a thiuram disulfide according to claim 1, in which the pouring of the dithiocarbamate salt composition into the reactor ends at the same time or before the pouring of the oxidizing composition.

10. A tetrabenzylthiuram disulfide obtainable by the preparation process according to claim 1, having a melting point of strictly greater than 135° C.

11. The use of the tetrabenzylthiuram disulfide according to claim 10 as a vulcanization accelerator, preferably an accelerator for the vulcanization of natural rubber, butadiene rubbers, butyl rubbers, EPDM (ethylene-propylene-diene monomer), latex, butadiene-acrylonitrile copolymer (or nitrile rubber or NBR) or styrene-butadiene copolymer (SBR).
